(19) United States
(12) Patent Application Publication
(10) Pub. No.: US 2003/0109464 A1
(43) Pub. Date: Jun. 12, 2003

(54) Galectins -1 and -4 in tumor development

(76) Inventors: Margaret E. Huflejt, La Jolla, CA (US); Valeri V. Mossine, Columbia, MO (US); Michael Croft, San Diego, CA (US)

Correspondence Address: Brenda J. Wallach, Morrison & Foerster LLP, Suite 500, 3811 Valley Centre Drive, San Diego, CA, 92130-2332, US

(21) Appl. No.: 10/262,017

(22) Filed: Sep. 30, 2002

Related U.S. Application Data
(60) Provisional application No. 60/326,137, filed on Sep. 28, 2001.

(51) Int. Cl. A61K 31/7012; A61K 31/7008; G01N 33/574

(52) U.S. Cl. 514/42; 514/53; 435/7.23

(57) ABSTRACT

Methods of prognosis and of prophylactic and therapeutic treatment of tumors based on the involvement of galectin-1 and galectin-4 in tumor development are described.

### CROSS-REFERENCE TO RELATED APPLICATIONS

This application claims benefit under 35 U.S.C. § 119(e) of provisional application Serial No. 60/326,137 filed 28 Sep. 2001. The contents of this application are incorporated herein by reference.

### STATEMENT OF RIGHTS TO INVENTIONS MADE UNDER FEDERALLY SPONSORED RESEARCH

This invention was made, in part, with support from the United States government. The United States government has certain rights in this invention.

### TECHNICAL FIELD

The invention is related to methods for prognosis of tumor development and for developing therapeutic compounds to inhibit tumor growth. More specifically, the invention concerns the involvement of galectins-1 and -4 in tumor development. The invention also concerns compounds useful for the inhibition of galectin-1 or galectin-4 mediated tumor growth.

### BACKGROUND ART

Galectins are members of a family of highly homologous, multifunctional, soluble animal lectins that bind carbohydrates containing terminal β-galactose moieties. Considerable attention has been paid to elevated expression levels of various galectins in the context of certain conditions. For example, galectin-1 appears to be associated with the inflammatory response as set forth in U.S. Pat. Nos. 6,054,315; 5,948,628; and 6,225,071. Galectin-1 was shown to be preferentially expressed in the more invasive parts of xenographs by Belot, R. S., et al., Glia (2000) 33:241-255. The expression of galectin-1 is also elevated in other cancers, including prostate carcinoma (Van den Brule, F. A., et al., J. Pathol. (2001) 193:88-87) and in various other cytomas and blastomas (Camby, I., et al., Brain Pathol. (2001) 11:12-26. Both galectin-1 and galectin-3 have been reported to be associated in some way in tumor development and metastasis. For example, Berberat, P. O., et al., J. Histochem. Cytochem. (2001) 49:539-549 report that galectin-1 and galectin-3 are expressed at higher levels in pancreatic cancer samples than in normal controls. Tinari, N., et al., Int. J. Cancer (2001) 91:167-172 report that both galectin-1 and galectin-3 bind to separate sites on a glycoprotein 90K which was described as a tumor-secreted antigen and found to have immunostimulatory activity. The proteins do not contain signal sequences; however, they can be exhibited at the cell surface and interact with matrix proteins. The overexpression of galectin-4 in breast cancers has been reported by Huflejt, M. E., et al., Proc. Am. Assoc. Canc. Res. (1997) 38:267 and in PCT publication WO 98/22139 published 28 May 1998, both incorporated herein by reference.

Glycoproteins have generally been recognized as cancer antigens, for example,, the Thomsen-Friedenreich antigen has been shown to be important in the adhesion of human breast and prostate cancers to the endothelium. This antigen is a simple mucin-type disaccharide, Galβ1-3GalNAc, which is expressed on the outer cell surfaces of T cell lymphomas and most human carcinomas, including breast and prostate. Glinsky, V. V., et al., Cancer Res. (2001) 61:4851-4857; Glinsky, V. V., et al., Cancer Res. (2000) 60:2584-2588. Indeed, a number of patents and patent applications filed by Glinskii are directed to methods of inhibiting cell adhesion, inducing apoptosis, and suppressing cancer metastasis using glycosylated amino acids or peptides. U.S. Pat. Nos. 5,629,412 and 5,864,024 disclose and claim such treatments where the composition utilized is a polypeptide having one or more amino acids, one of these amino acids being linked to a carbohydrate to form a Schiff base, an N-glycoside, an ester, or an Amadori product. The contents of these applications were published as PCT publications WO 96/01639 and WO 98/23625. These documents do not identify the targets or counterparts of these agents. In addition, PCT application WO 99/53930 describes similar activities of glycosylated amines, including some instances where a diamine is substituted with two separate carbohydrate moieties.

Although it is apparent that galectins are generally associated with tumor development and metastasis, the specifics of this association and the nature of the galectins involved is far from certain. For example, Huflejt, M. E., et al., J. Biol. Chem. (1997) 272:14294-14303 note that the localization of galectin-3 and galectin-4 in adenocarcinoma cells is widely different. Galectin-4 is localized at sites of cell adhesion, whereas galectin-3 is not. U.S. Pat. No. 6,423,314 indentifies particular amino acid sequences present on tumor cells that bind galactose.

Immunosuppressive properties of galectin-1 have been observed in connection with autoimmune diseases in animal models. See, for example, Levi, G., et al., Eur. J. Immunol. (1983) 13:500-507 (myasthenia gravis); Offner, H., et al., J. Neuro. Immunol. (1990) 28:177-284 (experimental allergic encephalitis); and Rabinovich, G. A., et al., J. Exp. Med. (1999) 190:385-398 (collagen-induced arthritis). Galectin-1 also induces apoptosis of T cells in vitro. See Perillo, N. L., et al., Nature (1995) 378:736-739; Perillo, N. L., et al., J. Exp. Med. (1997) 185:1851-1858.

Thus, the art recognizes that galectins in general have something to do with cancer, with cell adhesion, and with apoptosis, but the particulars of the involvements of any individual galectins are not well understood or characterized. The present invention focuses on two specific galectins, galectin-1 and galectin-4 and their involvement in cancer development, and in particular in the development of breast cancer. The invention also provides carbohydrate bearing compounds as antitumor agents; U.S. Pat. Nos. 5,895,784 and 5,834,442 discloses modified proteins as antitumor agents.

### DISCLOSURE OF THE INVENTION

The invention has several aspects, all related to the role of galectin-1 and/or galectin-4 in the development of cancer, especially breast cancer.

In one aspect, the invention is directed to a method to identify a subject who will develop malignant tumors especially of the breast, which method comprises assessing the level and distribution of galectin-4 in tissue of a subject, wherein said subject has been determined not to comprise malignant breast tumor cells; and

observing the presence or absence of clusters of elevated concentrations of galectin-4 in said tissue;

wherein the presence of said clusters identifies that individual as having a high probability for the development of malignant tumors, especially breast tumors.

In another aspect, the invention is directed to a method to inhibit the growth and/or metastasis of a tumor in a subject, especially a breast tumor, which method comprises administering to a subject in need of such treatment an amount of galectin-4 or a binding domain thereof effective to inhibit said growth and/or metastasis. In this embodiment, the galectin-4 is believed to act as a decoy with respect to endogenous materials that would ordinarily act as effectors on said galectin-4.

In a third aspect, the invention is directed to a method to inhibit the growth and/or metastasis of a tumor, especially a breast tumor, in a subject, which method comprises

determining the presence or absence or galectin-4 in the malignant tissues of said subject; and

administering to a subject whose tissues exhibit elevated levels of galectin-4 an effective amount of a therapeutic compound which binds to and/or inhibits the activity of galectin-4.

All of the foregoing aspects apply to galectin-1 as well.

In one embodiment, the therapeutic compounds are amino acids or polypeptides coupled to one or more sugars, preferably disaccharides. The treatment set forth above may be an adjuvant to additional methods to treat said tumor. In still another embodiment, the invention is directed to a method to identify anti-tumor compounds which method comprises assessing the ability of candidate compounds to bind to galectin-1 or galectin-4, whereby compounds which are found to bind galectin-1 and/or galectin-4 are identified as anti-tumor compounds.

In still another aspect, the invention is directed to a method to effect immunosuppression in a tumor-bearing subject, which method comprises administering to a tumor-bearing subject in need of such immunosuppression an amount of galectin-1 sufficient to effect said immunosuppression.

### BRIEF DESCRIPTION OF THE DRAWINGS

FIGS. 1A-1C show the encoding nucleotide sequence, the amino acid sequence and sites of binding amino acids in galectin-4; FIGS. 1D-1E show corresponding data for human galectin-1.

FIG. 2 shows the structures of several compounds described herein: lactulose-L-leucine; fructose-D-leucine; and dilactulose-hexamethylenediamine (L2hmda).

FIGS. 3A-3D show representative patterns of galectin-1 expression in human breast tissues.

FIGS. 4A-4F show representative patterns of galectin-4 expression in human breast tissues.

FIGS. 5A-5E show various patterns of galectin-4 expression in human breast tissue.

FIGS. 6A and 6B show the effect of transfection with an expression system for galectin-4 on the tumor cell line MDCK.

FIGS. 7A-7E show, graphically, data describing the ability of several amino sugars to bind galectin-1.

FIGS. 8A-8D show the ability of two different amino sugars tested to bind galectin-4.

FIGS. 9A and 9B show the ability of various amino sugars to inhibit the binding of galectin-1 to the antigen 90K (FIG. 9A) or to laminin (FIG. 9B).

FIGS. 10A-10C show the immunosuppressive effect of galectin-1 and the ability of galectin-1 to effect apoptosis, as well as the ability of certain amino sugars to affect these activities.

FIG. 11 compares the ability of various galectin inhibitors to block the development of breast cancer in transgenic mice.

### MODES OF CARRYING OUT THE INVENTION

Many of the aspects of the invention involve the use of recombinant galectin-4 or recombinant galectin-1 and antibodies raised to them. These materials are available in the art. The genes encoding both of these proteins have been cloned and antibodies have been raised. E. coli cells modified to produce human galectin-1 are described by Cho, M., et al., J. Biol. Chem. (1995) 270:5128-5206. cDNA encoding full-length human galectin-4 may be amplified using the full length sequence at accession number U82953 (GenBank™/EBI Data Bank) as described in Huflejt, M., et al., J. Biol. Chem. (1997) 272:14294-14303, or using the partial sequence of accession No. AA054456 found at the world wide web address ncbi.nlm.nih.gov/Entrez/. The galectin-4 coding sequence may be amplified using standard PCR techniques. Restriction sites permitting facile introduction into expression vectors is routine. These proteins may be produced using standard recombinant techniques. Further, preparation of anti-serum to galectin-4 has been described by Bresalier, R. S., et al., Cancer (1997) 80:776-787; Oda, Y., et al., J. Biol. Chem. (1993) 268:5929-5939. Recombinant means of production of both galectin-1 and galectin-4 are thus standard in the art and obtaining antibodies thereto employs standard techniques as well.

As used herein, “antibodies” refers to immunospecific immunoglobulins or portions thereof, however made, including monoclonal antibodies, fragments of monoclonal antibodies such as Fab, Fab′ or F(ab′)₂ fragments as well as single-chain recombinantly produced antibodies (Fv) and forms of these which are chimeric or modified to assume characteristics of a particular type of subject, such as humanized antibodies.

One aspect of the invention is grounded in enhanced knowledge of the metabolic and anti-metabolic role of galectins-1 and -4, especially with regard to their involvement in cancer, especially breast cancer.

In a first aspect, it has been found that the presence of high levels of either galectin-1 or galectin-4 in biopsied samples of subjects who are shown to have only benign cells is predictive for the later development, within 1-5 years, typically, of malignancies. Typically, in the case of galectin-4, this elevated expression is observed in small groups of cells, designated “hot spots” within the benign tissues. This is particularly documented in breast tumors. In one study, described below, 26 such subjects were biopsied and surveyed. Of these 26, 9 had hot spots with elevated levels of galectin-4; 8 of these progressed to malignancy within five years. The 17 subjects whose biopsies did not exhibit galectin-4 hot spots were free of tumors after that time. Thus, it is believed that the presence of galectin-4 hot spots in benign tissue, especially breast tissue, from normal subjects is predictive of later development of malignancy, thus permitting therapeutic or prophylactic intervention prior to this development. In addition, the presence of galectin-4 in lymph nodes is an indication that lymph nodes should be excised as a precaution. Similar results are found with respect to galectin-1.

In one embodiment of the method of this aspect of the invention, a biopsy sample is obtained and treated histologically according to standard procedures. If desired, samples can be preserved by formalin fixation and embedding in paraffin. In a typical illustrative procedure, the biopsy itself or, preserved biopsy sections which have been deparaffinized, are first heat treated and then quenched for endogenous peroxidase activity by treatment with peroxide and nitrite. After blocking non-specific binding, sections are incubated with antibodies immunoreactive with galectin-1 or galectin-4 and the complexed antibodies are detected using standard ELISA techniques, or any other immunodetection technique. If desired, background stain may enhance the image. While the foregoing is a typical procedure, any histological procedure designed to detect the presence of galectin-1 and/or galectin-4 could be substituted. A variety of such procedures is known in the art.

While histological techniques are convenient, alternative methods for identifying the presence of galectin-4 (or galectin-1) in benign or malignant tissues may also be used. In addition to biopsies, suspensions obtained by fine-needle biopsies—i.e., cellular preparations, may be obtained and treated to analyze them for the presence of these proteins. These cellular preparations can be obtained directly as fine-needle biopsies, or through ductoscopy, breast lavage, or lavage of the lymph nodes. The materials may be extracted and analyzed using standard analytical techniques for galectin-4 and/or galectin-1. These analytical techniques, performed on extracts, include, without limitation, sandwich immunological assays, electrophoretic assays, and the like.

Further, the analysis may be performed in situ by utilizing targeting agents which couple to galectin-4 and/or galectin-1 and then detecting the characteristic conferred by the targeting agent. For example, the targeting agent, such as an antibody or antibody fragment may be coupled to a radionuclide and the location of the nuclides detected; alternatively, the targeting agent may contain a contrast agent for ultrasound or magnetic resonance imaging (MRI) or may confer a signal detectable by positron emission tomography (PET). A variety of in situ techniques may be used rather than subjecting the subject to a more invasive biopsy approach.

The methods of treatment described herein both as primary treatment and as an adjuvant to additional treatment modalities with respect to cancers, especially breast cancers, depend on the identification of individuals whose tumors express galectin-4 or galectin-1. Methods for identifying subjects whose tumors express this protein are standard in the art and include the in situ techniques and techniques performed on biopsies such as those described above. It does not appear that galectin-4 or galectin-1 is secreted, so biopsy or in situ detection is preferred to analysis of body fluids.

A subject identified as bearing a tumor which expresses galectin-4 or galectin-1 may then be treated according to the methods of the invention. These methods are applicable as well to subjects with benign cell masses that express this protein, since, as noted above, the presence of galectin-4 or galectin-1 is prognostic for the development of malignancy.

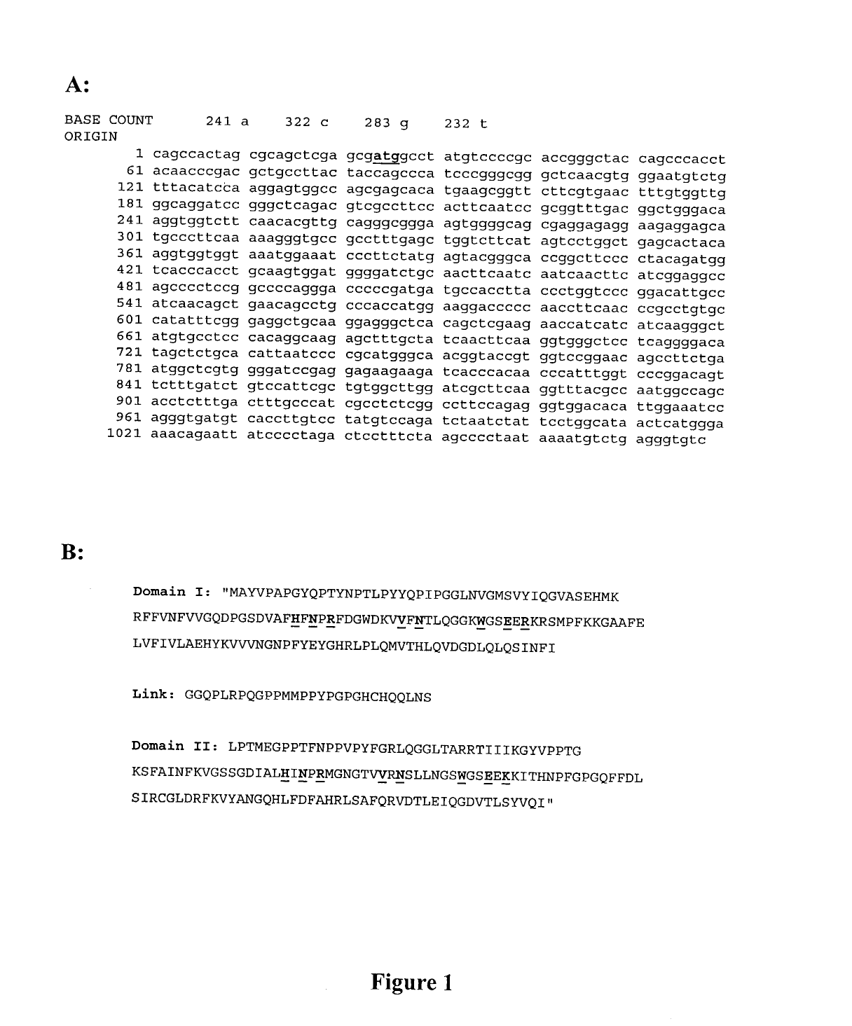

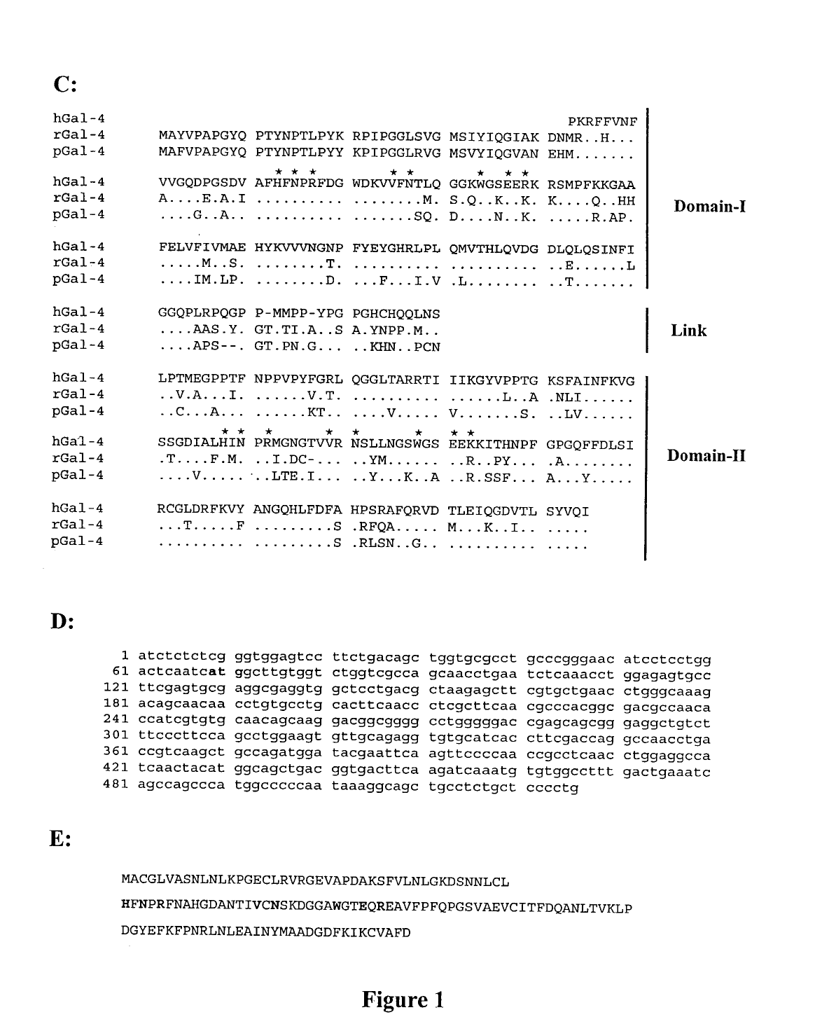

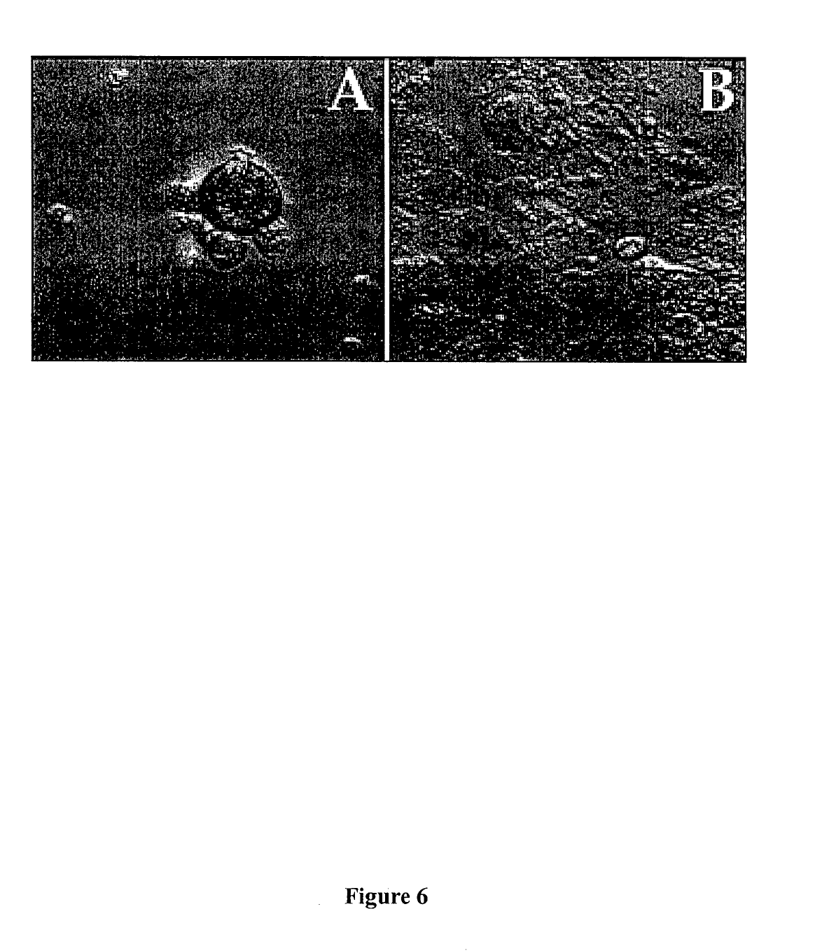

One approach is to supply galectin-4 itself, or a portion thereof that is able to bind its endogenous ligand as a decoy to prevent the undesired biological activity of galectin-4. Galectin-4 is a 36 kD protein which contains two tandem carbohydrate binding domains in a single polypeptide as described by Oda, Y, et al., J. Biol. Chem. (1993) 268:5929-5939. Each of the domains binds to a carbohydrate target; the presence of two domains permits simultaneous binding of two ligands containing different carbohydrate chains. Each carbohydrate binding domain is approximately 15 kD; useful portions of galectin-4 for use in the present invention would include individual domain 1 or domain 2 or those portions of the domains which contain the amino acid residues responsible for binding to the carbohydrate target. FIG. 1A shows the nucleotide sequence (start codon highlighted) encoding the amino acid sequence of human galectin-4. FIG. 1B shows the human protein with binding residues in bold, and FIG. 1C shows the location of the binding domains in human, rat and porcine galectin-4 as well as the amino acid residues responsible for binding which are marked with an asterisk (*). The segments of domains 1 and 2 which contain the asterisked sequences of FIG. 6C will be useful as decoys as well as will longer forms of these proteins. Proteins containing these binding domains which further contain heterologous amino acid sequence at the N- and/or C-terminus such that the additional sequence does not interfere with activity are useful as well. Further, the binding domains may be supplied coupled to other materials for ease of administration or alternative activity so long as the additional moieties do not interfere with the ability of galectin-4 to bind its biologically significant target.

Accordingly, suitable fragments useful in the invention would include those comprising the amino acid sequences

IFNPRFDGWDKVVFN(T/S)(L/M/Q)Q(G/S/D)G(K/Q)WG(S/K/N)EE(R/K)K or N(I/M)NPR(M/I/L)(G/T)(N/D/E)(G/C)(T/I)VVRNS(L/Y)(L/M)NG(S/K)WG(S/A)EE(K/R)K. These sequences are composites of those responsible for the binding of domain I and domain II respectively in the galectin-4 derived from human, rat and pig as shown in FIG. 1C. For use in intact organisms, the sequence characteristic of the organism is preferred. Thus, for example, for use in humans, the option at most or all of the indicated positions will be that of the human protein.

The encoding nucleotide sequence and amino acid sequence of galectin-1 are also known in the art. The coding sequence for human galectin-1 is set forth in FIG. 1D—the ATG start codon is bolded. FIG. 1E shows the binding domain (galectin-1 has only one) with critical residues bolded., Thus, a useful fragment would include the residues LHFNPRFNAHGDANTIVCNSKDGGAWGTEQRE which may be bracketed by heterologous sequence. Thus, in a manner similar to that described with respect to galectin-4, decoys comprising galectin-1 or one or more binding domains thereof may also be employed.

Proteins containing tandem copies of the binding domains of galectin-4 and/or galectin-1, including chimeras, may be employed. The required fragments described are approximate and 1-5 additional amino acids from the galectin sequences may also be included. Alternatively, an entire domain or the entire galectin may be used.

The galectin-4 or galectin-1 decoys can be administered in suitable pharmaceutical formulations and using routes of administration suitable for administration of proteins. Such routes include injection, transmembrane or transmucosal administration, transdermal administration, appropriately formulated oral administration and the like. Techniques for preparing pharmaceutical compositions appropriate for peptides and proteins is found, for example, in Remington's Pharmaceutical Sciences, latest edition, Mack Publishing Co., Easton, Pa., incorporated herein by reference.

Alternatively to supplying galectin-4 (or galectin-1) based peptides or proteins as decoys, individuals identified as harboring tumors expressing galectin-4 (or galectin-1) are also treated by administering compounds that bind galectin-4 (or galectin-1), thus preventing interaction of galectin-4 (or galectin-1) with its endogenous ligand. Such compounds can readily be identified by standard screening procedures which can be used to identify compounds from combinatorial libraries or individually synthesized compounds which are capable of binding this protein. The galectin-4 or galectin-1 for use in this assay may be produced recombinantly and displayed on host cells or may be purified and coupled to a solid support, either covalently or noncovalently or may be used in a homogeneous assay. Any standard assay for detection of binding is useful in this regard. A number of homogeneous assays are known which rely on interaction of labels associated with either or both of the candidate compound and the target galectin-4 or galectin-1 as well as heterogeneous assays such as those employing immobilized galectin-4 or galectin-1 or immobilized compounds. One particularly preferred assay, illustrated below, is the surface plasmon resonance based assay marketed by BIAcore, Inc., Uppsala, Sweden. Other assays, of course, can also be used.

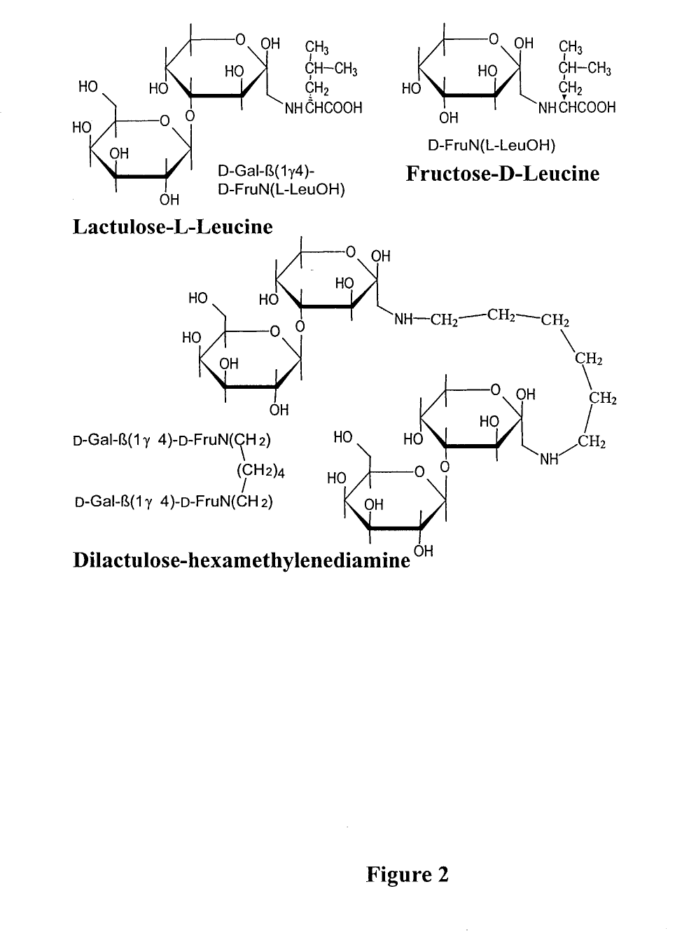

One class of compounds that provides candidates with a high probability of success is that of the glycoamines such as those described in the patents and applications attributed to Glinskii, described above and incorporated herein by reference. These compounds include amino acids or other moieties coupled as glycoamines to carbohydrate residues. Typical structures of such compounds are shown in FIG. 2 which includes the structure of fructose-D-leucine (FDL), lactulose-L-leucine (LL), and dilactulose-hexamethylenediamine (L2hmda). According to the results described herein, particularly preferred embodiments of compounds that bind galectin-4 and galectin-1 are those with multiple carbohydrate substitutions, such as the dilactulose-hexamethylenediamine illustrated. It is even more preferable to utilize compounds which have three or four or more glycoamine linkages. Also preferred are embodiments which include disaccharides as the glycoamines, especially derivatives of galactose and fructose. These compounds are available in the art as described in the Glinsky documents set forth above.

Thus, in one aspect, the invention is directed to a method to ameliorate the symptoms of or inhibit the development of a malignant tumor in a subject comprising cells that express galectin-4 which method comprises identifying the presence of galectin-4 in the subject's tissue and administering to said subject an glycoamine wherein said glycoamine comprises at least two disaccharides coupled through amino groups to a multivalent entity. Preferred embodiments include derivatives of alkylene diamines or alkylene triamines. Preferred disaccharide residues include lactose, lactulose, PGal-pGal, and the like.

Similarly, galectin-1 has been shown to bind substrates of this type; those subjects harboring cells that express galectin-1 can be similarly treated. Alternative agents can also be identified in a manner analogous to that set forth above for galectin-4 by screening compounds from combinatorial libraries or individually synthesized compounds.

It should be noted that the above documented treatments (either using galectin-4 or galectin-l peptides as decoys or using agents which bind to galectin-4 or galectin-1) may be used alone or in combination with alternative treatments such as chemotherapy, surgery, immune system enhancement, and the like. Thus, they are useful as treatments in their own right as well as as adjuvants to alternative treatments.

The resultant of treatment is generally described as inhibiting tumor growth and/or metastasis. The growth of a tumor is indicated by a number of phenomena, in particular, the presence or enhancement of angiogenesis, immunosuppression of the host—i.e., the inability of the immune system to attack the cancer itself, and the absence of conditions of apoptosis in the tumor cells. Any of these can be used as a measure of this inhibition of growth.

The invention is also directed to methods to enhance immune response in tumor-bearing subjects by administering compounds that specifically bind galectin-1. It is demonstrated hereinbelow that galectin-1 is immunosuppressive in this context and thus administration of compounds which bind to this target will enhance immune response to the tumor.

The following examples are intended to illustrate but not to limit the invention.

### Preparation A

### Production of Galectin-4 in E. coli

cDNA encoding full-length human galectin-4 was amplified from an EST clone (accession number AA054456), with the following primers: 5′-ACTGATATCATGGCCTATGTCCCCGCACCG-3′; and 5′-TCAGAATTCTTAGATCTGGACATAGGACAA-3′, which introduced EcoRV and EcoRI sites at the 5′ and 3′ ends, respectively. The PCR product was digested with EcoRV and EcoRI, ligated into bacterial expression plasmid pET28c in the restriction sites of the blunt-ended NcoI and EcoRI, and sequenced to confirm identity. This bacterial expression construct was transformed into BL21 (DE3)pLysS E. coli strain. For recombinant protein production, cultures were induced at O.D.₆₀₀ 0.5 with 0.4 mM IPTG and grown for 5 more hours. Cells were lysed by sonication in PBS, insoluble particles removed by centrifugation and cleared cell lysates were applied directly to a lactosyl sepharose column for affinity purification as described (Huflejt, M. E., et al., J. Biol. Chem. (1997) 272:14294-14303). 5 mM β-mercaptoethanol and 3 mM PMSF was present at all stages of protein purification.

### EXAMPLE 1

### Prognosis of Breast Cancer Development in Normal Subjects

Formalin-fixed, paraffin-embedded human breast tissues were obtained from tissue files of the Scripps Green Hospital, La Jolla, Calif. 5 μm-thick paraffin sections were deparaffinized in a series of xylenes through ethanol solutions to double distilled H₂O. To detect galectin-1, deparaffinized sections were heat-treated in a microwave oven in 10 mM citrate buffer pH 6.0 (Shi, S. R., J. Histochem. Cytochem., (1991) 39:741-748). Endogenous peroxidase activity was quenched by incubation in 3% H₂O₂/0.1% NaN₃/0.05% Tween-20 and non-specific antibody binding was blocked by incubation in 10% normal goat serum.

Sections were incubated with primary antibodies for 1 hour, rinsed in PBS, incubated with HRP-conjugated secondary antibodies (Bio-Rad, Hercules, Calif.) for 30 min., and the color reaction (amber) was developed with the Liquid DAB Substrate (BioGenex, San Ramon, Calif.). After counterstaining nuclei with hematoxylin (blue), sections were dehydrated and mounted in Permount (Fisher, Dallas, Tex.). Preimmune rabbit sera were used as negative control.

Primary antibodies were polyclonal rabbit anti-rat galectin-1 antiserum, a generous gift of Dr. Douglas N. Cooper (University of California, San Francisco), and rabbit polyclonal anti-rat galectin-4 antiserum (Huflejt, M. E., et al., J. Biol. Chem. (1997) 272:14294-14303). Specificities were confirmed by immunoblotting with purified human recombinant galectins and with the whole cell lysates.

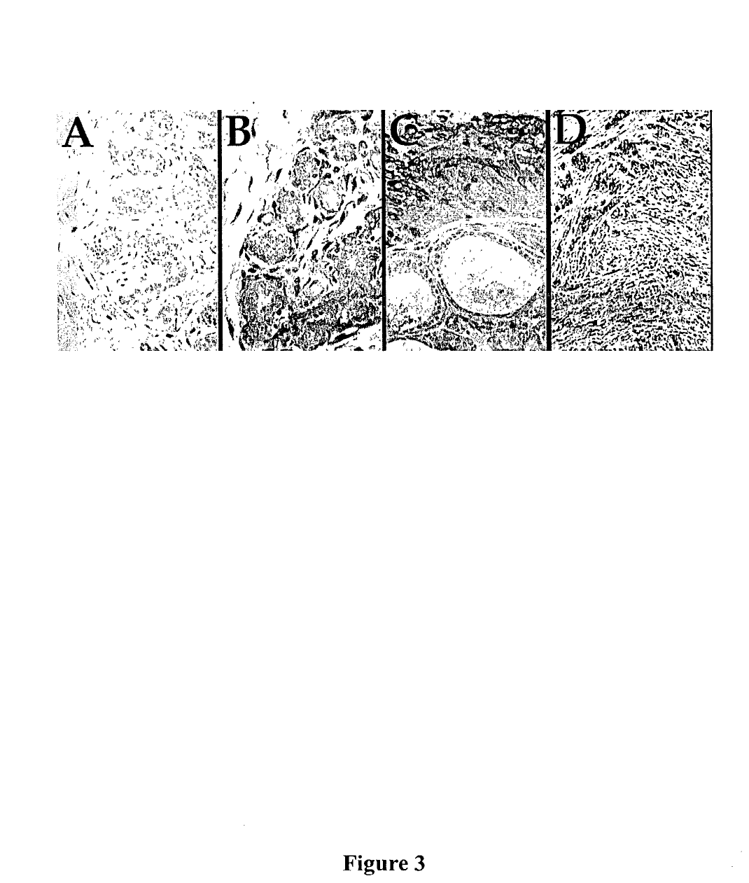

Typical results when the tissues were stained for galectin-1 expression are shown in FIGS. 3A-3D. FIG. 3A shows the results for normal reduction mammoplasty; FIG. 3B shows a morphologically normal lobule adjacent to a DCIS component; FIG. 3C shows the border between fibrocystic and malignant components and FIG. 3D shows the results where there is a malignant tumor. As shown, galectin-1 was absent in normal epithelium but expressed at high levels in most types of malignant tissue. Galectin-1 was also overexpressed in non-epithelial tissue components such as blood vessels and fibroblasts in a sub-population of patients with benign breast tumors.

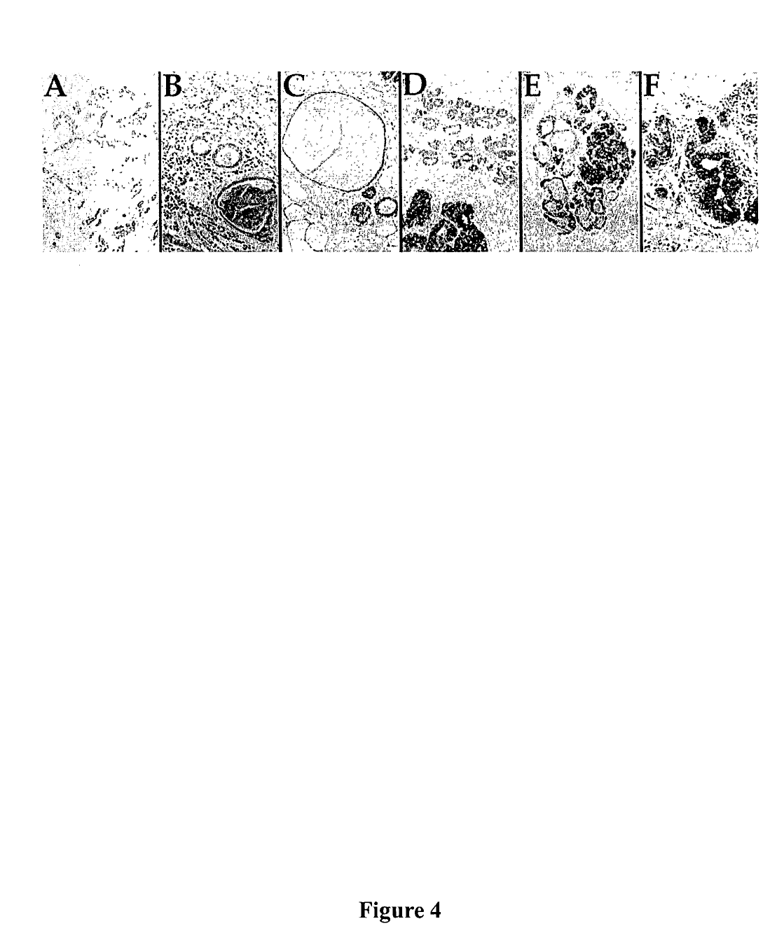

FIGS. 4A-4F show the results when galectin-4 is targeted. FIG. 4A is a normal reduction mammoplasty; FIG. 4B is of a hyperplasia without a typical component; FIG. 4C shows a small “hotspot” adjacent to fibrocystic non-proliferative ducts; FIG. 4D shows the border between a DCIS component and a morphologically normal lobule; FIG. 4E shows a DCIS component with enlarged individual cells overexpressing gal ectin-4; and FIG. 4F shows a typical “hotspot” of galectin-4 concentration estimated to be within the range of 4-8 μM as determined by immunoblotting breast cancer tissue extracts followed by densitometry using recombinant galectins as standards.

Twenty-six samples, described above, were obtained from subjects with benign hyperproliferation of breast tissue. Of these 26 biopsy samples, 9 showed the hotspots exemplified in FIG. 4F. Of these 9, 8 progressed to breast cancer within 1-5 years. None of the remaining 17 biopsied subjects, were no hotspots were found, progressed to cancer within this time period. Thus, expression of galectin-4 hotspots is an excellent means to identify subjects whose benign hyperproliferation will eventually become malignant.

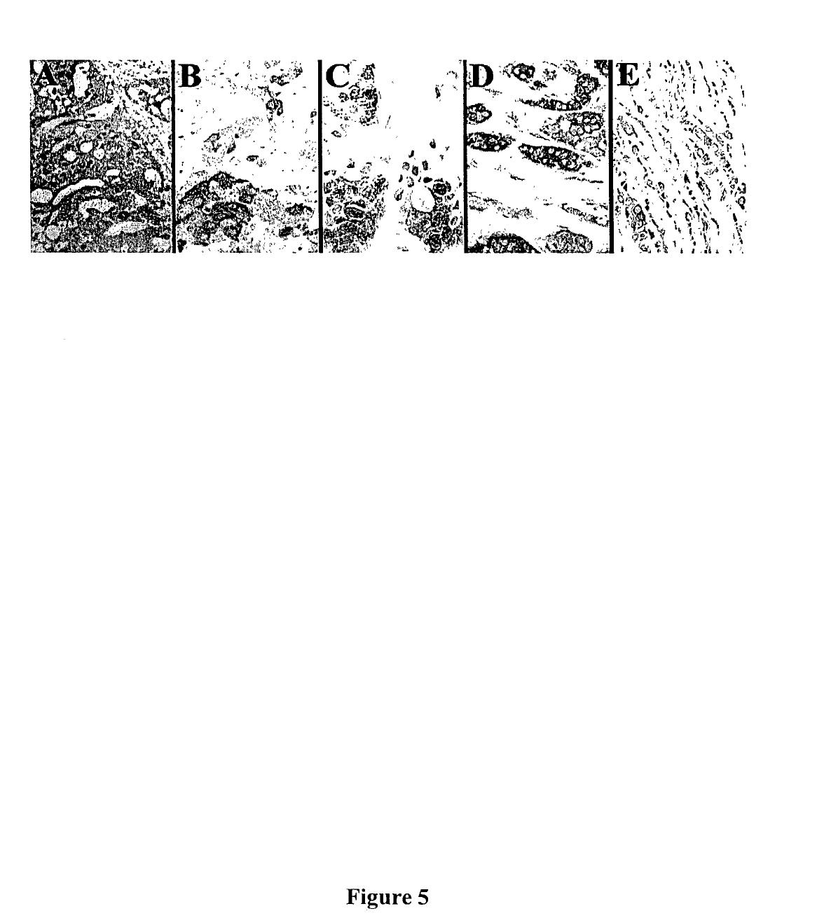

Additional examples of galectin-4 expression patterns in human breast tissues are shown in FIGS. 5A-5E; FIGS. 5A-5D are of ductal carcinomas and FIG. 5E is of a lobular carcinoma.

In another study using these techniques, a 10-year period was used with respect to diagnosis as benign in 1998. In 14 subjects where biopsies showed minimal or no galectin-4 expression, no malignancies developed. Sixteen subjects, however, whose tissue showed galectin-4 expression, 10 of the 16 showed malignancy within 5 years where hotspots were observed. Subjects who progressed to malignancy later, within 6-10 years, did not exhibit these hotspots.

In another study, 51 cases of benign tissues between 1985 and 1997 were studied. Of 26 cases that did not progress to malignancy, 25 showed minimal of no galectin-4 expression; 1 case showed the presence of 1 small hotspot. Of the 25 remaining subjects who progressed, 7 cases showed multiple hotspots; the lack of hotspots in the remaining samples of patients who progressed to malignancy is believed due to the age of the samples themselves.

### EXAMPLE 2

### Anti-Apoptotic Activity of Galectin-4

MDCK cells, which do not natively express galectin-4 (they express galectin-3), were transfected with full-length galectin-4-cDNA in order to observe the effect of galectin-4 on these cells. A comparison of cells transfected to produce galectin-4 and wildtype cells which were mock transfected is shown in FIGS. 6A and 6B. These cells were cultured for 7 weeks in serum-free medium before these photomicrographs were taken.

MDCK cells transfected with galectin-4-cDNA can be maintained in serum-free medium for 7-9 weeks, while mock transfected MDCK cells die within 10 days. Thus, galectin-4 has apparently an anti-apoptotic effect.

### EXAMPLE 3

### Detection of Galectins in Breast Epithelial Cell Lines

Cell lines MCF-7, MCF-10A, T47-D, and ZR-75-1 were obtained from ATCC (Manassas, Va.). For in vivo labeling with [³⁵S] methionine/cysteine, cell cultures at approximately 80% confluence were used, and endogenous methionine and cysteine were depleted and metabolic labeling was performed as described in detail in (Huflejt, M. E., et al., J. Biol. Chem. (1997) 272:14294-14303). Galectins were affinity-purified from clear cell lysates on 1 ml packed vol. lactosyl sepharose column, and eluted fractions were separated on a 12% SDS-PAGE gel. The combined label incorporated into galectins was determined by scintillation counting of aliquots of eluted fractions, and total amount of protein-incorporated label was determined from the trichloroacetic acid-precipitated radioactivity in aliquots of clear cell lysates before affinity chromatography.

Proteins were visualized by autoradiography, and relative label incorporation into individual galectins was determined using Phosphoimager and ImageQuant analysis.

The results indicated that all of these cell lines constitutively express comparable levels of galectin-3 (0.1-0.2% total incorporated label) but very low levels of galectin-4 (0.01-0.02% of total incorporated label). Thus, the cell lines tested do not express galectin-4 at levels comparable to those obtained in cells obtained from biopsies.

The MCF-10A cell line, which is a benign phenotype shows only trace amounts of galectin-1, but the cell lines known to be metastatic, T47-D and ZR-71-1 show high levels of galectin-1 expression corresponding to 1.2% of total incorporated label, or 4-8 μM in agreement with the levels in biopsied tissues. It has been shown previously by the current applicants that the breast adenocarcinoma cell line MDA-MB-435 which is invasive overexpresses galectin-1. Glinsky, V. V., Cancer Res. (2000) 60:2584-2588.

### EXAMPLE 4

### Ability of Glycoamines to Bind Galectin-1 and Galectin-4

The synthesis and chemical structures of fructose-amino acids and lactulose-L-leucine (LL) have been previously described (Mossine, V. V., et al., Carbohydr. Res. (1994) 262:257-270), and in U.S. Pat. Nos. 5,629,412 and 5,864,024 as well as PCT publication WO 99/53930. Briefly, D-glucose or D-lactose and appropriate amino acids are conjugated via a reaction that involves Amadori rearrangement. The synthesis and characterization of other lactulose-amino acids and dilactulose-hexamethylenediamine (L2hmda) is described in detail by V. V. Mossine, et al., (in press).

Briefly, amino acids or 1,6-hexamethylenediamine were brought into a reaction with D-lactose, in presence of a catalyst, typically acetic or malonic acid, and a browning inhibitor, such as sodium bisulfite. Suspensions of the reactants in anhydrous methanol or methanol/glycerol were refluxed until near completion of the reaction, followed by removal of the solvent by evaporation. Lactulose-amino acids were isolated and purified by means of ion-exchange chromatography. These compounds were isolated as hygroscopic solids and their identity and purity were confirmed by elemental analysis, thin-layer chromatography, NMR, mass-spectrometry, and/or polarimetry.

Galectin binding affinity measurements were performed in a surface plasmon resonance (SPR) assay using BIAcoreX instrument (BIAcore, Inc, Uppsala, Sweden). PBS with 5 mM β-mercaptoethanol was used as running buffer for all experiments. Purified recombinant human galectin-1 and galectin-4 were immobilized at different surface densities on CM5 carboxymethyldextran chip (BIAcore) in the presence of 150 mM lactose, using standard amine coupling chemistry. Galectin immobilization was performed at 5 μl/min flow rate. To ensure the maximum possible immobilization of recombinant proteins, carboxylmethyldextran surface was activated with a 15 min pulse of a mixture of 0.05 M N-hydroxysuccinimide (NHS) with 0.2 M N-ethyl-N′-(dimethylaminopropyl) carbodiimide (EDC). Recombinant galectins, diluted to 30 μg/ml in 10 mM Na Acetate buffer (pH 4.8 for galectin-1 and pH 5.5 for galectin-4) were coupled to the surface during a 15-minute injection. Following galectin injection, remaining activated groups on the surface of the chip were deactivated with a 7 min pulse of 1 M ethanolamine hydrochloride, pH 8.5. Blank, unmodified CM5 surface was used as a negative control for the corrections for refractive index changes due to the presence of lactulosamines.

Binding of lactulosamines was performed at 80 μl/min flow rate, with injection times of 15 sec. At all concentrations tested, equilibrium binding levels were achieved instantaneously, as expected for lectin-carbohydrate interaction with very fast association and dissociation rates. Therefore, direct measurements of association and dissociation rate were not possible. Instead, the equilibrium binding levels were examined and determined the equilibrium binding constant using steady state affinity fitting model (B1Aevaluation software version 3.1).

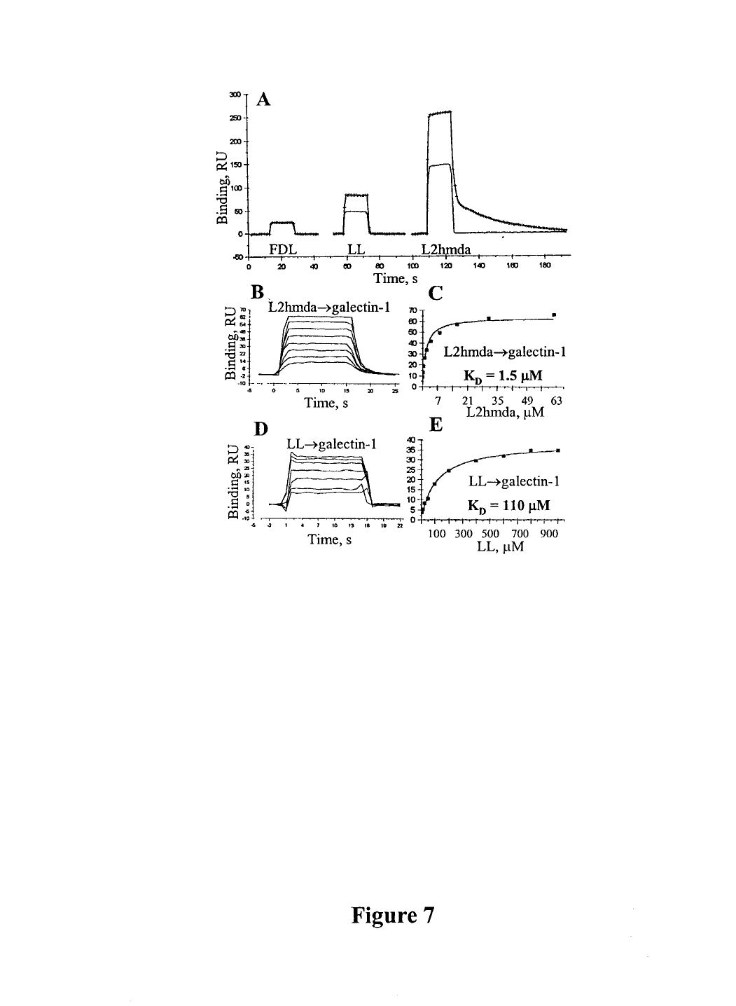

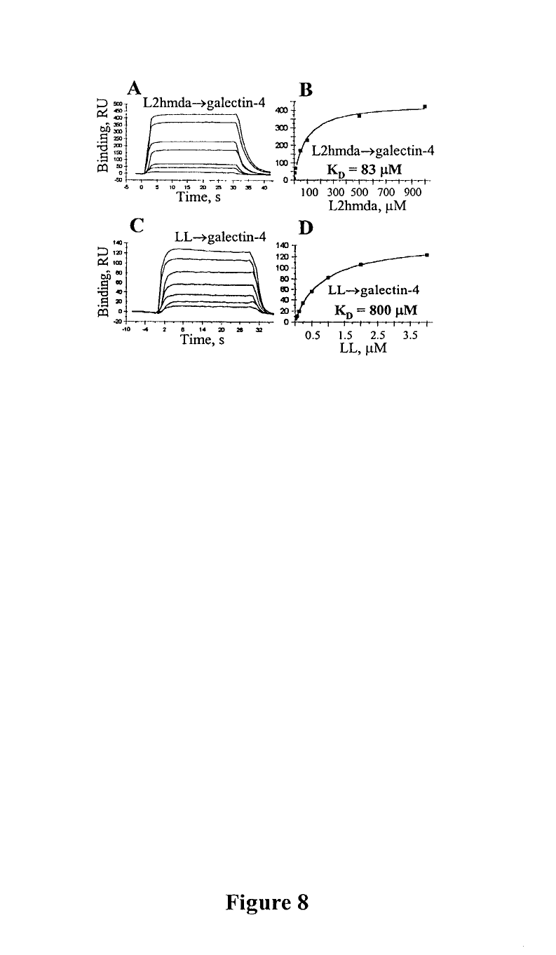

The results are shown in Table 1 and in FIGS. 7-8. Table 1 shows the affinity of various glycoamines as dissociation constants in units of micro-molar (μM). FIG. 7 shows typical patterns obtained as functions of time and concentration for galectin-1 and FIG. 8 shows comparable data for galectin-4.

It has been shown previously that fructose amines and lactulose-L-leucine (LL) block clonogenic growth and metastasis of breast cancer cells in a nude mouse model (Glinsky, G. V., et al., Cancer Res. (1996) 56:5319-5324) and that LL blocks adhesion of cancer cells to endothelium (Glinsky, V. V., et al., Cancer Res. (2001) 61:4851-4857).

In the SPR assay, LL and other lactulose-amino acids could bind galectin-1, while fructose-amino acids did not, as shown in FIG. 7A for LL and fructose D-leucine (FDL). Multimeric ligands are known to have higher affinity for their cognate proteins. In agreement with this general phenomenon, L2hmda interaction with galectin-1 was significantly stronger when compared to LL and to other lactulose amines. Plotting the equilibrium binding levels versus the analyte concentration, and determining the Kd value by steady-state analysis determined the affinities of galectin interactions with lactulosamines. FIGS. 7B-7E show the binding of LL and L2hmda to galectin-1. In all cases, the association rate and the dissociation rate of binding of lactulosamines to galectin-1 was too fast for direct fitting. An exception was binding of L2hmda to galectin-1 where the dissociation phase could be analyzed. Best fitting was obtained for a two-component dissociation model, where fast component (40% of overall rate) was k_{off} 0.16-0.2 s⁻¹ (corresponding to a half-life of 3 seconds), and a slow component (60% of overall rate) with a k_{off} 0.036 s⁻¹ (corresponding to a half-life of 19 seconds). A weighted average of the two components of dissociation rate gives a half-life of 7 seconds. The K_{D} value for galectin-1 with LL is about 110 μM and with L2hmda, about 1.5 μM.

FIG. 8 shows the binding of LL and L2hmda to galectin-4. The analysis of the data for galectin-4 is complicated by the fact that the full-length galectin-4 has two carbohydrate-binding domains, and each of them may have different affinities for various lactulosamines was obtained. However, even with these limitations, a ranking of affinities of lactulosamines for galectin-4. As shown, K_{D} for galectin-4 with LL is about 800 μM and with L2hmda is about 80 μM.

Table I presents a compilation of affinity measurements for binding of lactulosamines and some fructose amines to human galectin-1 and galectin-4. The values presented are average of three to four measurements performed during independent experiments. The differences between individual measurements were within 5-10% for K_{D} values obtained for galectin-1, and within 20% for K_{D} values obtained for galectin-4. In general, most affinity values for interactions of single lactose-containing compounds with galectin-1 fell between 0.1 and 0.2 mM, what suggests that at least on the level of direct binding, contributions of different amino acids to the stabilization of binding interaction can not be identified.

TABLE I
Equilibrium binding affinity of glycoamines for galectins-1 and -4. NB, non-binder

| Compound | Galectin-1 binding, K_{D}, μM | Galectin-4 binding, K_{D}, μM |
|---|---|---|
| Dilactulose-hexamethylenediamine | 1.5, 1.8, 3.5 | 78, 83, 105, 115 |
| Lactulose-L-alanine | 150 | 1490, 1720 |
| Lactulose-D-alanine | 133 | 1010 |
| Lactulose-L-leucine | 113, 200 | 505, 800, 1006 |
| Lactulose-D-leucine | 128, 223 | 770, 880, 1090 |
| Lactulose-glycine | 102 | 790 |
| Lactulose-L-isoleucine | 129 | 550 |
| Lactulose-L-proline | 70 | 530 |
| Lactulose-L-threonine | 376 | 3830 |
| Lactulose-L-valine | 158 | 719, 730 |
| Lactulose-L-methionine | 127 | 1270 |
| Lactulose-L-histidine | 191 | 2280 |
| Lactulose-L-phenylalanine | 200 | 1090, 1260 |
| Lactulose-L-GABA | 106 | 1040 |
| Fructose-D-leucine | NB | NB |
| β-lactose | 269, 351 | 807, 1070, 1350 |
| Maltose | NB | NB |
| Sucrose | NB | NB |

As shown in Table 1, maltose, sucrose, and fructose coupled to leucine are unable to bind either galectin-1 or galectin-4. However, P-lactose is able to bind both proteins. As expected, the multi-glycosylated dilactulose hexamethylenediamine is superior in binding to both galectin-1 and galectin-4.

### EXAMPLE 5

### Binding of Galectin-1 to Immobilized Glycoproteins

Human 90K/MAC-2BP (thereafter called 90K) was purified from transfected EBNA-293 cells (Iurisci, I., et al., Clin. Cancer Res. (2000) 6:1389-1393). Laminin was obtained from Boehringer Mannheim GmbH (Germany). The ELISA for galectin binding has been previously described (Tinari, N., et al., Int. J. Cancer (2001) 91:167-172). Briefly, Nunc Maxisorb microtiter wells were coated with 0.5 μg/well of 90K or laminin. Recombinant galectin-1 (2.5 μg/ml or 0.17 μM) was diluted in PBS with 0.1% Tween-20 and added to wells coated with glycoproteins, and galectin-1 binding was detected with anti-galectin-1 rabbit antiserum. In binding-inhibition experiments, recombinant galectin-1 was diluted in PBS-0.1% Tween-20 containing indicated concentration of lactulosamines or lactose.

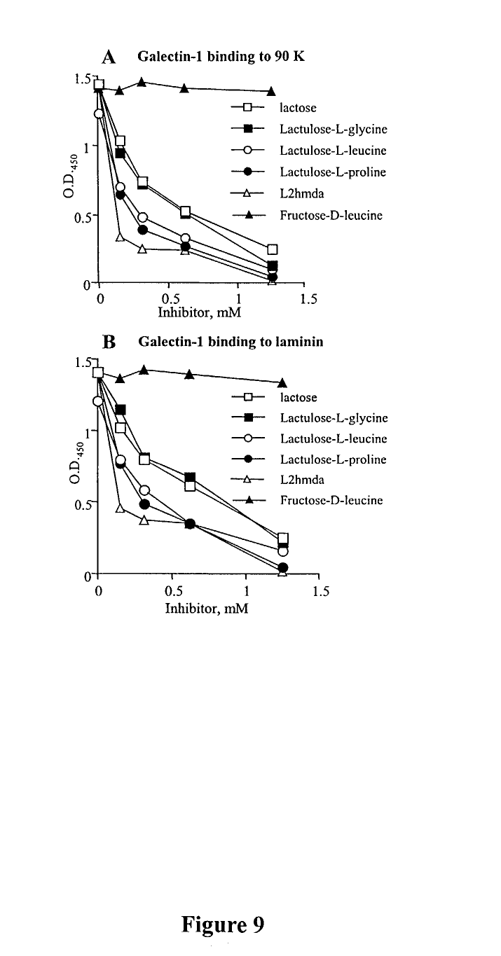

It is known that binding of galectin-1 to the extracellular matrix component laminin is important in cell adhesion. Binding of galectin-1 to 90K and to laminin was inhibited by lactulosamines and by lactose, but not by fructose amines, as shown in FIG. 9. The affinity ranking for lactulosamine-galectin-1 interactions in ELISA followed ranking observed in SPR binding assays, with divalent L2hmda being the most potent inhibitor of galectin-1 binding to both 90K and laminin: at 156 μM, L2hmda inhibited about 80% binding of galectin-1 to 90K and about 70% binding to laminin, whereas at the same concentration LL and lactulose-L-proline inhibited binding to 90K by about 50% and 40% to laminin, and lactulose-L-glycine and lactose only by about 20% to 90K and 10% to laminin.

It has previously been shown that galectin-1 binds 90K and that the complex augments cell aggregation (Tinari, N., et al., Int. J. Cancer (2001) 91:167-172. It is also known that galectin binds laminin as an extracellular matrix component. As shown in FIG. 9 above, the results obtained for fructose-D-leucine are surprising in view of the demonstration that fructose amines reduce breast cancer metastasis in the nude mouse model.

### EXAMPLE 6

### Glycoamines Overcome the Immunosuppressive Effect of Galectin-1

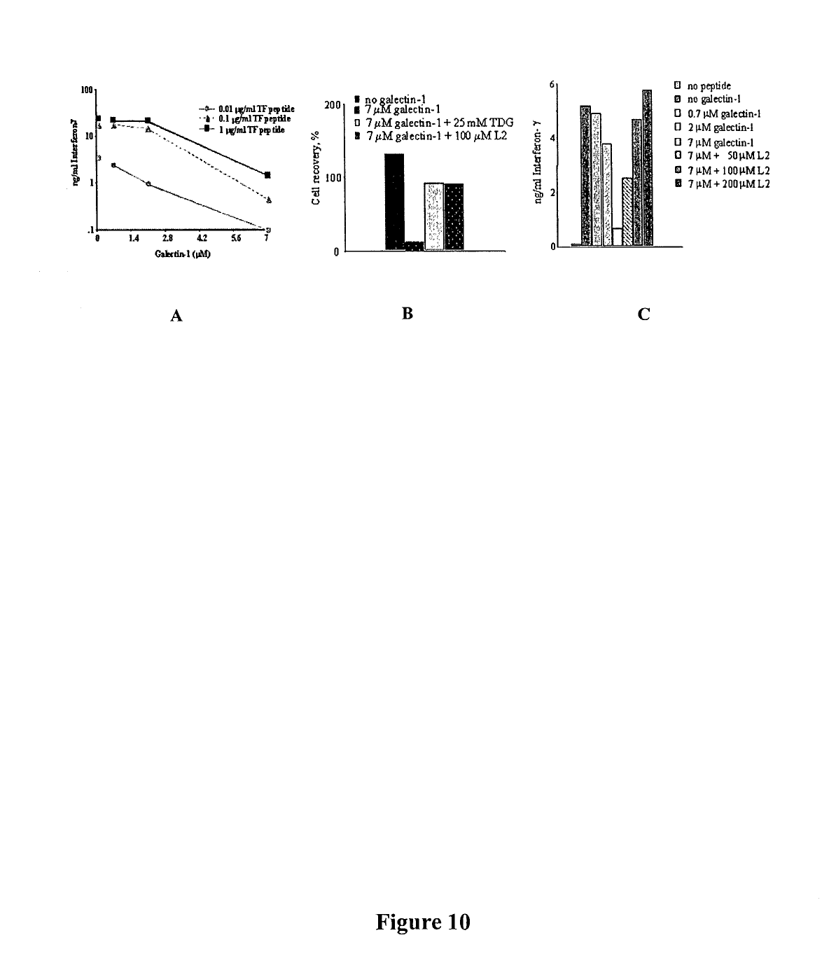

Treatment in vitro of CD4+ and CD8+ T cells with recombinant soluble galectin-1 inhibits their survival and ability to secrete cytokine, such as interferon-γ and IL-2. Although it has been known in the art that galectin-1 is immunosuppressive in general, the immunosuppressive properties of this protein have not hitherto been established or suggested in the context of cancer. In FIG. 10A, cells are stimulated by TF peptide and the levels of interferon-y produced measured in nanogram/milliliter (ng/ml). As shown, galectin-1 inhibits interferon-y secretion in a dose-dependent manner. FIG. 10A shows interferon-y production by a CD8+ CTL clone after stimulation with increasing concentrations (0.01-1 μg/ml) of a peptide of the Thompson-Friedenreich (TF) antigen presented on irradiated splenocytes. IFN-γ, measured by ELISA assay in supernatants taken 48 hr after stimulation, was suppressed in a dose dependent fashion by galectin-1 (0-7 μM). Similar data were seen when another cytokine, IL-2, was measured (not shown).

FIG. 10B shows that L2hmda as well as thiodigalactolate (TDG, a known galectin inhibitor) are able to restore the ability of CTL's to proliferate. The ability of L2hmda to restore this activity is especially significant since TDG, a known galectin inhibitor, is toxic. However, L2hmda is not toxic. FIG. 10B shows that addition of galectin-1 results in death of T-lymphocytes. The CD8+ CTL clone was stimulated with TF peptide for 4 days and cell survival (% of starting number) measured by enumerating viability by trypan blue exclusion. Galectin-1 resulted in death of 90% of the T cells over the 4 day period. In contrast, the galectin inhibitors thiodigalactolate (TDG) or L2 prevented the majority of cell death induced by galectin-1.

The effect of adding various concentrations dilactulose hexamethylenediamine is shown in FIG. 10C. As shown, enhancing the levels of L2hmda restores the interferon γ secretion to normal when galectin-1 is present at 7 μM and when L2hmda is present at 200 μM. FIG. 10C demonstrates that galectin-1 mediated suppression of IFN-γ production by the CTL was completely prevented by administering increasing concentrations (50-200 μM) of the galectin inhibitor L2hmda.

### EXAMPLE 7

### Effect of L2hmda on Tumor Treatment

Neu mice were inoculated on Day 0 with 10⁶ N202.1A tumor cells. The animals were divided into four groups of four mice each. On Day 7, groups 2 and 4 were subjected to dendritic cell therapy which involved immunization with 10⁶ dendritic cells pulsed with apoptotic N202.1A cells and intraperitoneal injections of recombinant IL-2 (10⁴ I.U./injection) from Day 10 to Day 7. Groups 3 and 4 received injections of 100 μl of 100 μM L2hmda daily.

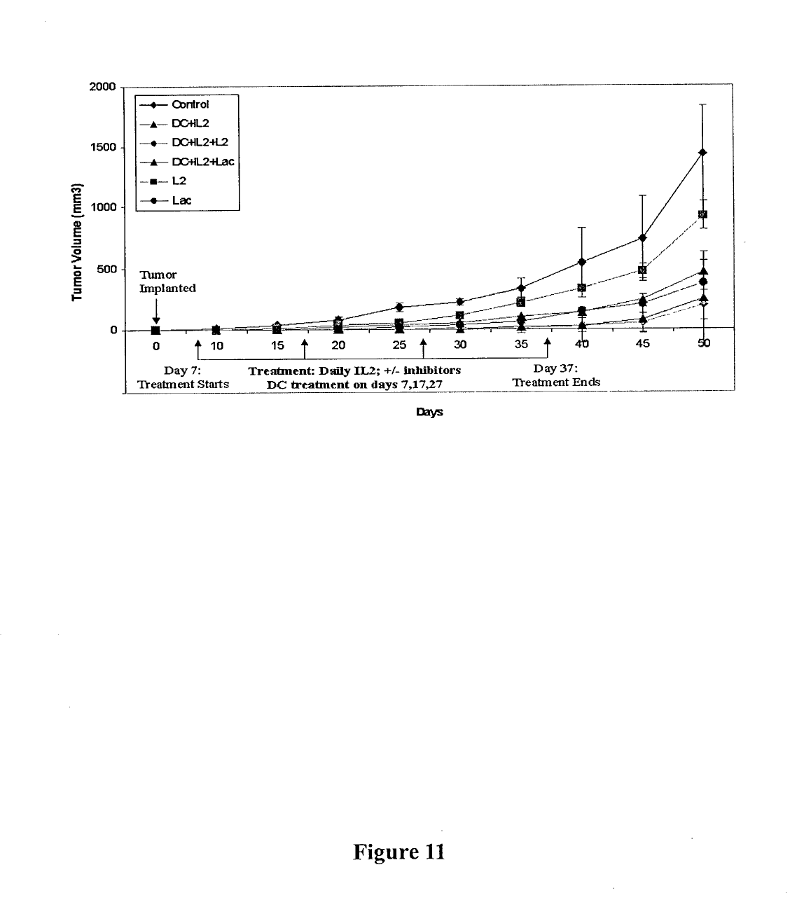

The results are shown in FIG. 11. As shown, the tumor volume of control group 1 increased dramatically over the first 25 days of the experiment; combination treatment with dendritic cells therapy and L2hmda was superior to treatment with either alone; indeed, very little tumor volume enhancement over 25 days was observed in this group 4. Similar effects were observed when lactose was substituted for L2hmda.

1. A method to identify a subject who will develop malignant tumors, which method comprises assessing the level and distribution of galectin-4 in tissue of a subject, wherein said tissue has been determined not to comprise malignant tumor cells; and
observing the presence or absence of clusters of elevated concentrations of galectin-4 in said tissue;
wherein the presence of said clusters identifies that individual as having a high probability for the development of malignant tumors.

2. The method of claim 1, wherein the tissue is breast tissue.

3. A method to inhibit the growth and/or metastasis of a tumor in a subject, which method comprises administering to a subject in need of such treatment an amount of galectin-4 or a binding domain thereof effective to inhibit said growth and/or metastasis.

4. The method of claim 3, wherein said tumor is a breast tumor.

5. A method to inhibit the growth and/or metastasis of a tumor in a subject, which method comprises
determining the presence or absence of galectin-4 in the malignant tissues of said subject; and
administering to a subject whose tissues exhibit elevated levels of galectin-4 an effective amount of a compound which binds to and/or inhibits the activity of galectin-4.

6. The method of claim 5, wherein said tumor is a breast tumor.

7. The method of claim 5, wherein said compound is a glycoamine.

8. The method of claim 7, wherein the glycoamine is an organic moiety comprising at least two amino groups, wherein said amino groups are coupled to disaccharides.

9. The method of claim 8, wherein said organic moiety comprises at least three amino groups, and wherein said amino groups are coupled to disaccharides.

10. A method to identify compounds useful as candidates for tumor therapy, which method comprises
contacting a test compound with galectin-4 or a cell expressing galectin-4 and
determining whether said test compound binds to said galectin-4 protein or said cell, whereby a compound which binds to said galectin-4 protein or cell is identified as a candidate compound for antitumor therapy.

11. A method to identify a subject who will develop malignant tumors, which method comprises assessing the level and distribution of galectin-1 in tissue of a subject, wherein said tissue has been determined not to comprise malignant tumor cells; and
observing the presence or absence of clusters of elevated concentrations of galectin-1 in said tissue;
wherein the presence of said clusters identifies that individual as having a high probability for the development of malignant tumors.

12. The method of claim 11, wherein the tissue is breast tissue.

13. A method to inhibit the growth and/or metastasis of a tumor in a subject, which method comprises administering to a subject in need of such treatment an amount of galectin-1 or a binding domain thereof effective to inhibit said growth and/or metastasis.

14. The method of claim 13, wherein said tumor is a breast tumor.

15. A method to inhibit the growth and/or metastasis of a tumor in a subject, which method comprises
determining the presence or absence or galectin-1 in the malignant tissues of said subject; and
administering to a subject whose tissues exhibit elevated levels of galectin-1 an effective amount of a compound which binds to and/or inhibits the activity of galectin-1.

16. The method of claim 15, wherein said tumor is a breast tumor.

17. The method of claim 15, wherein said compound is a glycoamine.

18. The method of claim 17, wherein the glycoamine is an organic moiety comprising at least two amino groups, wherein said amino groups are coupled to disaccharides.

19. The method of claim 18, wherein said organic moiety comprises at least three amino groups, and wherein said amino groups are coupled to disaccharides.

20. A method to identify compounds useful as candidates for tumor therapy, which method comprises
contacting a test compound with galectin-1 or a cell expressing galectin-1 and
determining whether said test compound binds to said galectin-1 protein or said cell, whereby a compound which binds to said galectin-1 protein or cell is identified as a candidate compound for antitumor therapy.

21. A method to effect immunosuppression in a tumor-bearing subject, which method comprises administering to a tumor-bearing subject in need of such immunosuppression an amount of galectin-1 sufficient to effect said immunosuppression.

22. A method to stimulate the immune system of a subject, which method comprises administering to a subject in need of such stimulation an amount of a compound which binds to galectin-1 sufficient to effect such stimulation.

23. The method of claim 22, wherein said compound is a glycoamine.

24. The method of claim 23, wherein the glycoamine is an organic moiety comprising at least two amino groups, wherein said amino groups are coupled to disaccharides.

25. The method of claim 24, wherein said organic moiety comprises at least three amino groups, and wherein said amino groups are coupled to disaccharides.
